(19) United States
(12) Patent Application Publication
(10) Pub. No.: US 2013/0058998 A1
(43) Pub. Date: Mar. 7, 2013

(54) ENCAPSULATED N-ACETYLMANNOSAMINE OR N-ACETYLNEURAMINIC ACID TO INCREASE SIALYLATION

(71) Applicants: Marjan Huizing, Bethesda, MD (US); Tal Yardeni, Tel-Aviv (IL); Christopher M. Jay, Carrollton, TX (US); Nancy Smyth Templeton, Carrollton, TX (US); William A. Gahl, Kensington, MD (US)

(72) Inventors: Marjan Huizing, Bethesda, MD (US); Tal Yardeni, Tel-Aviv (IL); Christopher M. Jay, Carrollton, TX (US); Nancy Smyth Templeton, Carrollton, TX (US); William A. Gahl, Kensington, MD (US)

(73) Assignee: United States of America as represented by the Secretary, Department of Health and Human Services

(21) Appl. No.: 13/605,946

(22) Filed: Sep. 6, 2012

Related U.S. Application Data
(60) Provisional application No. 61/531,934, filed on Sep. 7, 2011.

(51) Int. Cl. A61K 31/7028; A61P 25/28; A61P 35/00; A61K 9/127; A61P 13/12

(52) U.S. Cl. 424/450; 514/42

(57) ABSTRACT

A method for treating a disorder characterized by hyposialylation in a subject that includes administering to the subject an effective amount of (i) a composition comprising N-acetylmannosamine, or a derivative thereof, encapsulated within a liposome, or (ii) a composition comprising N-acetylneuraminic acid, or a derivative thereof, encapsulated within a liposome, wherein the liposome comprises 1,2-dioleoyl-3-trimethylammonium-propane and cholesterol.

### CROSS-REFERENCE TO RELATED APPLICATION

This application claims the benefit of U.S. Provisional Application No. 61/531,934, which was filed on Sep. 7, 2011, and is incorporated herein by reference in its entirety.

### BACKGROUND

Hereditary inclusion body myopathy (HIBM; OMIM 600737) is a rare autosomal recessive neuromuscular disorder. Argov et al., Neurology 60, 1519-1523 (2003); Eisenberg et al., Nat Genet. 29, 83-87 (2001); Griggs et al., Ann Neurol 38, 705-713 (1995). The disease usually manifests approximately between 20 and 30 years of age with foot drop and slowly progressive muscle weakness and atrophy. Histologically, it is associated with muscle fiber degeneration and formation of vacuoles containing 15-18 nm tubulofilaments that immunoreact like β-amyloid, ubiquitin, prion protein and other amyloid-related proteins. Askanas et al. Curr Opin Rheumatol 10, 530-542 (1998); Nishino et al. Acta Myol 24, 80-83 (2005); Askanas, et al. Ann Neurol 34, 551-560 (1993); Argov et al. Curr Opin Rheumatol 10, 543-547 (1998). Both weakness and histological changes initially spare the quadriceps. However, the disease is relentlessly progressive, with patients becoming incapacitated and wheelchair-confined within one to three decades.

### SUMMARY

One embodiment disclosed herein is a method for increasing sialic acid in a subject in need thereof comprising administering to the subject an effective amount of (i) a composition comprising N-acetylmannosamine, or a derivative thereof, encapsulated within a liposome, or (ii) a composition comprising N-acetylneuraminic acid, or a derivative thereof, encapsulated within a liposome, wherein the liposome comprises 1,2-dioleoyl-3-trimethylammonium-propane and cholesterol, to thereby increase sialic acid levels in the subject.

Another embodiment disclosed herein is a method for treating a disorder characterized by hyposialylation in a subject comprising administering to the subject an effective amount of (i) a composition comprising N-acetylmannosamine, or a derivative thereof, encapsulated within a liposome, or (ii) a composition comprising N-acetylneuraminic acid, or a derivative thereof, encapsulated within a liposome, wherein the liposome comprises 1,2-dioleoyl-3-trimethylammonium-propane and cholesterol.

The foregoing will become more apparent from the following detailed description, which proceeds with reference to the accompanying figures.

### BRIEF DESCRIPTION OF THE DRAWINGS

FIG. 1 shows the N-acetylneuraminic acid synthesis pathway. The biosynthesis of N-acetylneuraminic acid (NANA, Neu5Ac) starts with glucose in the cytosol, which is converted in several steps into Uridine diphosphate —N-acetylglucosamine (UDP-GlcNAc), serving as substrate for the bifunctional, key enzyme of sialic acid biosynthesis, UDP-GlcNAc 2-epimerase/ManNAc kinase (GNE). Hinderlich, et al, J Biol Chem 272, 24313-24318 (1997). The GNE epimerase catalytic activity epimerizes UDP-GlcNAc to ManNAc, after which the ManNAc kinase catalytic activity phosphorylates ManNAc to ManNAc-6-Phospate. Two other enzymes convert ManNAc-6P further to NANA. NANA (or any other sialic acid) is activated into CMP-sialic acid. CMP-sialic acid can then be utilized in the Golgi complex by sialyltransferases for the sialylation of glycans, which are mostly expressed on the cell surface. HIBM patients have defects in GNE, resulting in decreased production of NANA. Eisenberg, et al. Nat Genet. 29, 83-87 (2001); Noguichi et al., J Biol Chem 279, 11402-11407 (2004); Huizing et al., Mol Genet Metab 81, 196-202 (2004); Ricci et al., Neurology 66, 755-758 (2006); Broccolini et al. Neurochem 105, 971-981 (2008); Tajima et al. Am J Pathol 166, 1121-1130 (2005). ManNAc (uncharged sugar) feeding increases NANA formation and sialylation (even if ManNAc kinase is defective, because other kinases can convert ManNAc to ManNAc 6P). Hinderlich et al., Biol Chem 382, 291-297 (2001)). NANA feeding itself can also increase sialylation, however NANA is negatively charged, making it harder to get into cells.

FIG. 2 is a graph depicting the survival of mutant HIBM Gne M712T mutated pups at P5 after different treatments (all IV deliveries were performed at P1). Without treatment >90% of mutant mouse pups die before postnatal day 3 (P3) (8% survived) because of severe glomerular disease (kidney failure) due to hyposialylation. We tested different treatment strategies and monitored survival (and sialylation status at P5). All wild type pups survived (100%) at P5 without treatment. Survival rates increased after oral (not liposome-embedded) ManNAc (48% survival) or oral NANA (10%) addition to drinking water. Intravenous (IV) injection of ManNAc itself (not liposome-embedded) did not significantly increase survival rate (8%). IV injection of ManNAc-Lipoplex (91% and 100% in 2 different mouse backgrounds) and NANA-Lipoplex (85%) significantly increased mutant survival rate at P5.

FIG. 3 is a graph showing average weight of HIBM Gne M712T mice by groups. Red: The weight of untreated wild type and heterozygous Gne M712T mice steadily increases as they age (data shown up to 31 weeks). Green: The weight of mutant mice, treated at P1 with ManNAc-Lipoplex to survive beyond P3, but then not received any other treatment is significantly decreased compared to the control group (Red). Purple: the weight of mutant mice, treated at P1, and then once every month with ManNAc-Lipoplex increased compared to untreated mutants (Green). Indicating rescue of muscle degeneration and/or rescued kidney function with treatment. Blue: Wild type and heterozygous mice that were treated with ManNAc-Lipoplex once a month did not significantly gain weight compared to the untreated control group (Red), indicating that ManNAc-Lipoplex treatment in unaffected animals does not result in increased weights.

FIG. 4 is a representation of lectin staining. Data is presented for treatment with 3 different lectins (specifically bind to sugars) that only recognize the endgroup to a glycan. SNA will only bind if sialic acid (SA) is present as an endgroup. PNA will only bind if galactose (Gal) is free as an endgroup, i.e., if sialic acid is not present. In normal sialylated cells, PNA will not/hardy bind. HPA will only bind to O-linked glycans if N-acetyl galactosamine (Ga1NAc) is free as an endgroup, if sialic acid (and galactose) is not present. In normal, sialylated cells, HPA will not/hardly bind.

(Apart from these 3 lectins, there is data (not presented) on staining with several other lectins, including WGA, VVA, MAL).

FIG. 5 shows the results of paraffin embedded slides of mouse kidneys treated with 3 lectins (green signals) and a nuclear dye (blue, to indicate presence of cells). Without treatment, mutant pups (−/−) show hyposialylated glomeruli, indicated by the green signals after HPA and PNA staining (absent in wild type pups (+/+)), and reduced SNA staining compared to +/+ pups. At P5, after ManNAc-Lipoplex treatment at P1, mutant pups are rescued from the hyposialylation (no PNA and HPA binding after treatment, increased SNA staining, similar to wild type). At older age without further treatment, kidneys show hyposialylation, illustrated by 6 month old untreated kidneys (note that these mice were ManNAc-Lipoplex treated once at P1 to let them survive beyond P3) which show significant HPA binding and reduced SNA binding (green signals). At older age after monthly ManNAc-Lipoplex treatment (shown at 10.5 mo), kidneys show improved/normal glomerular sialylation status (similar to unaffected +/− mice at this age), with absent HPA binding and increased SNA binding.

FIG. 6 shows the results of paraffin embedded slides of mouse kidneys treated with 2 lectins (green signals) and a nuclear dye (blue, to indicate presence of cells). Without treatment, mutant pups (−/−) show hyposialylated glomeruli, indicated by the green signals after HPA and PNA staining (absent in wild type pups (+/+)). At P5, after NANA-Lipoplex treatment at P1, mutant pups are rescued from the hyposialylation (no PNA and HPA binding after treatment, similar to unaffected heterozygote (+/−).

FIG. 7 depicts mouse kidney electron microscopy and Western blotting. Electron microscopy of the glomerular filtration apparatus in wild type (+/+) mice show nicely formed podocyte foot-processes (finger-like structures) that retain their shape because of negatively charged sialic acid groups on their cell surface. Mutant Gne M712T mice have hyposialylated glomeruli, and podocyte foot processes appear fused and flattened, resulting in filtration defects and renal failure and death before P3 in these pups, if untreated. Galeano et al. J Clin Inv 117, 1585-1594 (2007). After ManNAc-Lipoplex treatment (at P1), the shape of the foot processes is recovered/rescued (arrow), likely contributing to survival of these mice beyond P3. Podocalyxin (PDXN) is the most abundant, heavily sialylated glycoprotein in podocyte membranes. Western blotting of mouse kidneys at P2 shows an upward shift (less negatively charged) of PDXN signal compared to wild type (arrows at left). At P5, after treatment the sialylation on PDXN appeared to be (partially/completely) recovered, illustrated by the shift back down to (almost) normal size (arrows at right). Nephrin is a (not heavily) glycosylated protein important for glomerular filtration. Western blotting of mouse kidneys at P2 shows a downward shift (smaller molecular weight) of nephrin signal compared to wild type (arrows at left). At P5, after treatment the sialylation on nephrin appeared to be (partially/completely) recovered, illustrated by the shift back up to (almost) normal size (arrows at right).

FIG. 8 depicts mouse kidney Western blotting. Upper Panel: Western blotting with antibodies against Podocalyxin (PDXN), the most abundant, heavily sialylated glycoprotein in podocyte membranes. Western blotting of mouse kidneys at P2 shows an upward shift (less negatively charged) of PDXN signal compared to wild type (+/+) and heterozygous (+/−) kidneys. At P5, after treatment with NANA-Lipoplex at P1, the sialylation on PDXN appeared to be (partially/completely) recovered, illustrated by the shift back down to (almost) normal size in two mutant (−/−) mice kidneys at P5. Lower Panel: Western blotting with antibodies against Nephrin, a (not heavily) glycosylated protein important for glomerular filtration. Mutant mouse kidneys (−/−) at P2 shows a downward shift (smaller molecular weight) of nephrin signal compared to wild type (+/+) and heterozygous (+/−). At P5, after treatment with NANA-Lipoplex at P1, the sialylation on nephrin appeared to be (partially/completely) recovered, illustrated by the shift back up to (almost) normal size in two mutant (−/−) kidneys.

FIG. 9 shows lectin staining of mouse muscle (gluteus). Left Panels, without treatment: Mutant (−/−, affected) and heterozygous (+/−, unaffected) Gne M712T mice were injected with ManNAc-Lipoplex at P1 (to let the mutants survive beyond P3, but did not receive any treatment thereafter. Muscle (gluteus) slides of these mice at 6 and 7 months were stained with the lectins HPA and SNA. HPA staining (binds O-linked Ga1NAc when sialic acid is absent) shows a signal/binding in the mutant (−/−) muscle, but not in the unaffected (+/−). SNA staining (binds sialic acid) shows a signal in the unaffected (+/−), but hardly in the mutant (−/−) muscle. These findings indicate hyposialylation of mutant Gne M712T muscle tissue at older age (7 mo). Right Panels, with ManNAc treatment once a month: Muscles (gluteus) from 6-10 month old mice treated once a month with ManNAc-Lipoplex were stained with the lectins HPA and SNA. Both HPA and SNA staining in the mutant muscle staining showed similar staining pattern as the unaffected heterozygous (+/−), indicating that ManNAc-Lipoplex treatment once a month rescued their muscle hyposialylation, similar to the treatment effect in kidneys.

FIG. 10 depicts human kidney lectin staining. 40 human paraffin embedded kidney biopsy/autopsy slides were stained with the lectins HPA and SNA. Eight kidneys (20%) showed hyposialylation. A feature not previously recognized in such renal disorders. Such human renal disorders with hyposialylation may be good candidates for ManNAc-Lipoplex therapy. Shown are 3 representative examples of minimal change nephritis samples (left panels), where 2 samples (Patients 1 and 2) showed hyposialylation and one did not (Patient 3). Also shown are 3 representative examples of Lupus Nephritis samples (right panels), where 2 samples (Patients 11 and 12) showed hyposialylation and one did not (Patient 13).

Hyposialylation found in the following samples:
2 out of 5—Minimal change nephritis
2 out of 8—Lupus nephritis
2 out of 6—Focal Segmental Glomerulosclerosis
1 out of 5—Immune complex dependent glomerulonephritis
1 out of 4—IgA nephropathy
0 out of 4—IgM nephropathy
0 out of 4—Membranous glomerulopathy
0 out of 4—Others (incl. radiation nephritis, pheochromocytoma, renal cell carcinoma)

Total tested: 40 patients' samples

Total hyposialylated: 8 patients' samples

FIG. 11 shows slides from human muscle biopsies that were stained with: The lectins HPA and Jacalin (both only binding when sialic acid is absent from O-linked glycans): showing staining/binding to muscle cell membranes, indicating hyposialylation, in a HIBM patient (left panels), but not in his unaffected brother (right panels); and an antibody recognizing the polysialic acid (PSA) on PSA-NCAM, showed no staining in the HIBM muscle (left), but a strong signal in muscle of his unaffected brother (right). This indicates that the patient lacks the sialic acid groups on NCAM.

### DETAILED DESCRIPTION

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

“Administration” as used herein is inclusive of administration by another person to the subject or self-administration by the subject.

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

“Inhibiting” refers to inhibiting the full development of a disease or condition. “Inhibiting” also refers to any quantitative or qualitative reduction in biological activity or binding, relative to a control.

The term “subject” includes both human and veterinary subjects.

A “therapeutically effective amount” or “diagnostically effective amount” refers to a quantity of a specified agent sufficient to achieve a desired effect in a subject being treated with that agent. Ideally, a therapeutically effective amount or diagnostically effective amount of an agent is an amount sufficient to inhibit or treat the disease without causing a substantial cytotoxic effect in the subject. The therapeutically effective amount or diagnostically effective amount of an agent will be dependent on the subject being treated, the severity of the affliction, and the manner of administration of the therapeutic composition.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” is inclusive of inhibiting the full development of a disease or condition, for example, in a subject who is at risk for a disease, or who has a disease, such as cancer or a disease associated with a compromised immune system. “Preventing” a disease or condition refers to prophylactic administering a composition to a subject who does not exhibit signs of a disease or exhibits only early signs for the purpose of decreasing the risk of developing a pathology or condition, or diminishing the severity of a pathology or condition.

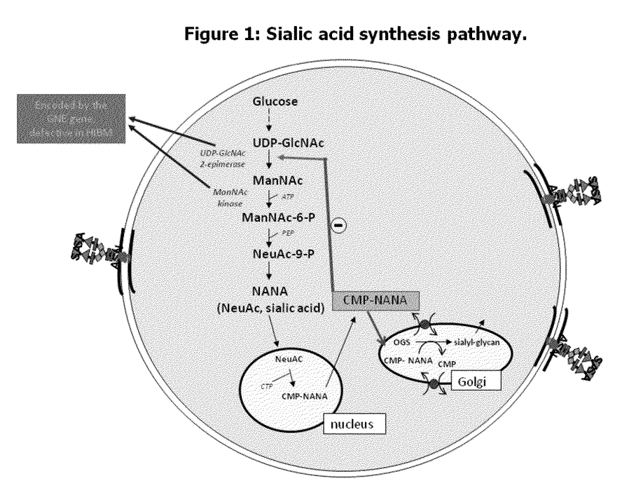

N-acetyl-D-mannosamine is a key compound in the sialic acid biosynthetic pathway (see FIG. 1). In particular, there is a regulated, rate-limiting enzymatic step in the pathway that leads to sialic acid formation, and this rate-limiting step gives rise to N-acetyl-D-mannosamine. Hence, once N-acetyl-D-mannosamine is formed or administered, no other enzymatic step leading to the formation of sialic acid is subject to feedback inhibition. Thus, administration of N-acetyl-D-mannosamine will lead to increased amounts of sialic acid. The structure of N-acetyl-mannosamine is shown below.

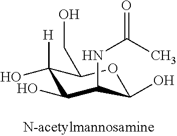

Therefore, administration of N-acetylmannosamine (ManNAc) and/or its derivatives promotes formation of sialic acid (N-acetylneuraminic acid) in a subject. Sialic acids are negatively-charged sugars found on many cellular and tissue components. For example, sialic acids are present on most cell surfaces, and on proteins and lipids and are involved in cell to cell interactions. Sialic acid-rich oligosaccharides on the glycoconjugates found on surface membranes help keep water at the surface of cells. The sialic acid-rich regions also contribute to creating a negative charge on the cells surface. Since water is a polar molecule, it is attracted to cell surfaces and membranes. Thus, sialic acids contribute to cellular hydration and fluid uptake. Sialic acid is also a vital component of many body fluids including, serum, cerebrospinal, saliva, amniotic, and mother's milk.

N-acetylmannosamine and derivatives thereof can be used for encapsulation by the liposomes disclosed herein. The structures of such N-acetylmannosamine derivatives are defined by Formula I.

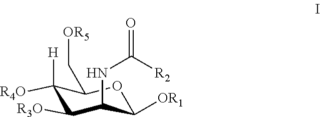

wherein:

R₁, R₃, R₄, or R₅ is hydrogen, lower alkanoyl, carboxylate or lower alkyl; and

R₂ is lower alkyl, lower alkanoylalkyl, lower alkyl alkanoyloxy.

The following definitions are used, unless otherwise described: Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to.

Lower alkyl refers to (C₁-C₆)alkyl. Such a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

Sialic acid is a generic term for the N- or O-substituted derivatives of neuraminic acid, a monosaccharide with a nine-carbon backbone. Sialic acid also is often used for the most common member of this group, N-acetylneuraminic acid (also referred to herein as “NANA” or “Neu5Ac”). NANA is the most abundant mammalian sialic acid, and the precursor of all other sialic acids. As used herein, “N-acetylneuraminic acid, or a derivative thereof,” refers to NANA and the family of sialic acids that comprises more than 50 members, differing in particular on the hydroxyl substituents of the common precursor NANA (Angata and Varki, Chem Rev 102, 439-469 (2002); Cohen and Varki. OM/CS 14, 455-464 (2010).

In certain embodiments, the liposome compositions disclosed herein may be a closed structure comprising an outer lipid bi- or multi-layer membrane surrounding an internal aqueous space. In particular, the liposomes form vase-like structures which invaginate their contents between lipid bilayers. The liposome compositions comprise 1,2-bis(oleoyloxy)-3-trimethylammonium-propane (DOTAP) and at least one cholesterol and/or cholesterol-derivative. In certain embodiments, the molar ratio of DOTAP to cholesterol is 1:1-3:1. The liposome compositions may be prepared by heating, sonicating, and sequential extrusion of the lipids through filters of decreasing pore size, thereby resulting in the formation of small, stable liposome structures as described, for example, in U.S. Pat. No. 6,413,544, which is incorporated herein by reference in its entirety.

In some embodiments disclosed herein, the methods are performed to treat a disease or condition. For example, such a disease may be a disorder characterized by hyposialylation. Examples of disorders that involve hyposialylation include muscular atrophy (Malicdan et al., Acta Myol 26, 171-175 (2007); Noguichi et al., J Biol Chem 279, 11402-11407 (2004); Huizing et al., Mol Genet Metab 81, 196-202 (2004); Ricci et al. Neurology 66, 755-758 (2006); Broccolini et al., Neurochem 105, 971-981 (2008); Tajima et al., Am J Pathol 166, 1121-1130 (2005)), kidney disease (Galeano et al. J Clin Inv 117, 1585-1594 (2007); Blau et al., Lab Invest 28, 477-481 (1973); Quatacker et al., Pathol Res Pract 182, 188-194 (1987); Coppo and Amore, Kidney Int 65, 188-194 (2004)), cancer tissue that is hyposialylated (Oetke, et al. Biochem Biophys Res Commun 308, 892-898 (2003); Suzuki et al. Int J Mol Med 22, 339-348 (2008); Ohyama, Int J Clin Oncol 13, 308-313 (2008)), and neurodegenerative disorders (involving amyloid accumulation in brain tissue), including Alzheimer's disease (Malicdan et al., Autophagy 3, 396-398 (2007)). Hyposialylation conditions that may be amenable to the treatments disclosed herein can be detected by several different methods. For example, lectin panels (described below in more detail in the Examples section) can be used to show overall hyposialylation, as demonstrated for mouse and human kidney and muscle hyposialylation. References to specific hyposialylated proteins that were found for each condition are noted below. Other methods to detect sialylation would be analytical sialic acid assays, however, overall sialylation is likely normal in most of these disorders, but specific glyco/sialoproteins/glycans are hyposialylated. This can be detected by Western blotting (as we showed for podocalyxin and nephrin for kidney disorders). Another method to detect sialylation defects is glycan profiling in tissues/samples.

In general, the types of muscular atrophies that can be treated by the disclosed methods and compositions are those caused by sialic acid deficiency. Examples of such muscular atrophy diseases and conditions include distal myopathy with rimmed vacuoles (DMRV or Nonaka myopathy, OMIM605820) and hereditary inclusion body myopathy (HIBM, OMIM600737).

In particular, the N-acetylmannosamine (or derivatives thereof)-liposome or N-acetylneuraminic acid (or derivatives thereof)-liposome compositions are useful therapeutic agents for increasing production of sialic acids in mammals, and such increased production of sialic acid has profound therapeutic benefits. Sialic acids are important for proper development and functioning of many organs and tissues, and a deficiency of sialic acid can give rise to many different types of diseases and conditions. Varki et al. Trends Mol Med 14, 351-360 (2008).

As shown herein, administration of the liposome compositions is useful for treating myopathies, muscular atrophy and/or muscular dystrophy (e.g., hereditary inclusion body myopathy (HIBM)) and kidney conditions and diseases (e.g., those involving proteinuria and/or hematuria).

Myopathies that can be treated with the present compositions and methods also include distal myopathy with rimmed vacuoles (Nonaka myopathy) and the muscular dystrophy hereditary inclusion body myopathy (HIBM, also called IBM2).

The compositions and methods are useful for treating certain kidney conditions and diseases, for example, those involving proteinuria and/or hematuria resulting primarily or secondarily from hyposialylation (lack of sialic acid). Thus, the present methods are effective for treatment of kidney disorders due to poor formation and/or function of membranes of the glomerular filtration apparatus. For example, kidney membranes that are affected by loss of sialic acid include the glomerular basement membrane and/or the podocyte membranes. Galeano et al. J Clin Inv 117, 1585-1594 (2007). Hence, the methods and compositions are useful for treating malformed or poorly functioning glomerular basement membranes and/or podocyte membranes. In general, the methods can increase sialylation of kidney podocalyxin, improve podocyte foot process morphology and/or improve glomerular basement membrane integrity.

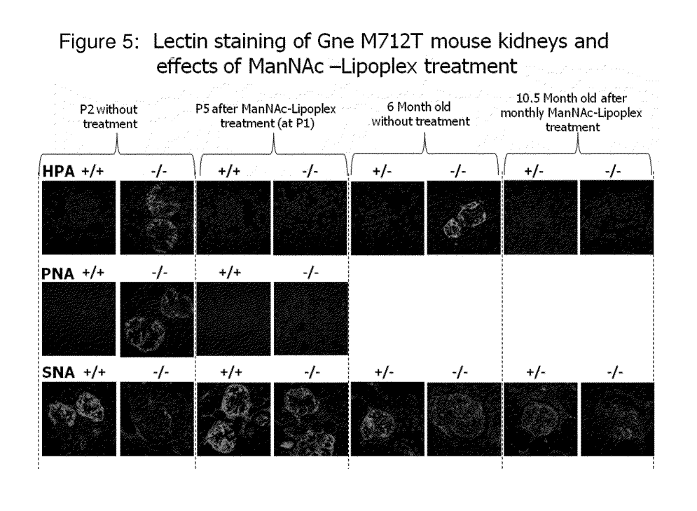

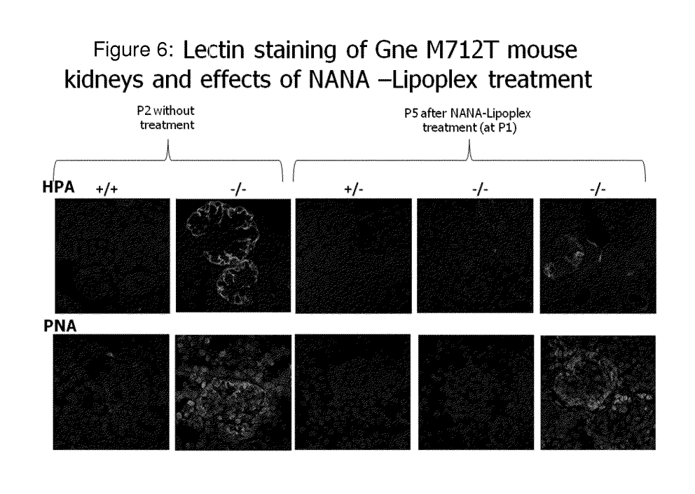

Thus, another embodiment disclosed herein is a method of treating a kidney disorder in a subject comprising administering a therapeutic amount of the N-acetylmannosamine (or a derivative thereof)-liposome composition, or the N-acetylneuraminic acid (or a derivative thereof)-liposome composition to the subject, wherein the kidney disorder involves proteinuria and/or hematuria, and a kidney biopsy shows hyposialylation (for example detected by a lectin staining panel, see FIGS. 5, 6, and 10).

Proteinuria involves leakage of protein from the blood into the urine. If the amount of protein in the urine is very high, this condition is often called nephrotic syndrome. While there may be many causes for nephritic syndrome, according to the invention at least one cause is a deficiency of sialic acid, which has a direct impact on the formation, structure and function of kidney glomeruli and the membranes associated therewith. Several types of diseases exhibit the symptoms of proteinuria, including high blood pressure, infections, reflux nephropathy, diabetes, various types of glomerulonephritis, including minimal change nephrosis. However, by improving the structure and function of nephron components that require sialic acid, the present compositions and methods can treat any of these diseases. Thus, for example, the presently disclosed methods and compositions dramatically improve kidney function by improving the structure and filtration properties of kidneys, thereby reducing the amount of protein in the urine and/or the severity or progression of proteinuria.

Hematuria simply means blood in the urine. The blood may be visible, so that the urine appears reddish or darker than normal (called gross hematuria). If the blood is invisible and is discovered only when a urine sample is examined in a laboratory urine test, the condition is called microscopic hematuria. In general, hematuria is more a symptom than a condition in itself, because it has many possible causes. A urinary tract infection, kidney or bladder stones, an enlarged prostate in men, cystitis (a bladder infection, usually in women) or bladder, kidney or prostate cancer can all cause hematuria. Other causes include injuries that result in bruised kidneys; sickle cell anemia and other abnormal red blood cell diseases; and certain medications, such as blood thinners (e.g., aspirin and some other pain relief medicines). More specific causes of glomerular basal membrane dysfunction, such as Alport disease, thin membrane disease, and IgA nephropathy, may particularly improve when the treatment methods described herein are employed. Thus, the compositions disclosed herein may be used to treat podocytopathies, minimal change nephrosis, focal and segmental glomerulosclerosis, membranous glomerulonephritis, and other forms of unexplained idiopathic nephrotic syndrome, as well as glomerular basement membrane diseases such as Alport disease and thin membrane disease. Such kidney disorders and conditions are sometimes characterized by segmental splitting of the glomerular basement membrane and/or podocytopathy due to disturbed polyanions on podocyte membranes, or to changes in the amount or charge (sialylation) of glomerular basement membrane components.

In general, the treatment methods disclosed herein involve administering to a subject a therapeutically effective amount of N-acetylmannosamine and/or a derivative thereof, or N-acetylneuraminic acid and/or a derivative thereof, as delivered by the liposome composition. Such a therapeutically effective amount is generally given daily for appropriate periods of time. Effective amounts are, for example, about 0.1 g/day to about 50 g/day, of about 0.2 g/day to about 25 g/day, from about 0.3 g/day to about 12 g/day, from about 0.4 g/day to about 10 g/day, from about 0.5 g/day to about 8 g/day, and from about 0.7 g/day to about 6 g/day. In certain embodiment, a therapeutically effective amount is 0.05 to 5, more particularly, 0.1 to 1, g/kg/day, which may be only injected once a month (or another timeframe such as once a week) in certain embodiments. Generally, the N-acetylmannosamine (and/or a derivative thereof)/liposome composition, or N-acetylneuraminic acid (and/or a derivative thereof)-liposome composition, is administered for periods of time sufficient to increase the amount of sialic acid in the subject and thereby achieve a therapeutic benefit. Therapeutic benefits that can be achieved by administration of N-acetylmannosamine and/or a derivative thereof, or N-acetylneuraminic acid and/or a derivative thereof, include improved kidney function, reduction in protein excretion in the urine, reduction in blood concentrations in the urine, increased sialylation of specific target glycoproteins (including podocalyxin and nephrin), fewer cystic tubular dilatations in the kidney cortex and in the kidney medulla, less fusion and flattening of the podocyte foot processes, greater number of open slit diaphragms in the kidneys, improvement in the “finger shaping” of the kidney foot processes, improved overall integrity of the glomerular basement membrane (GBM), increased GNE-protein expression and GNE-epimerase activities.

ManNAc is a ubiquitous but rare monosaccharide involved in a range of metabolic processes. It is uncharged and crosses membranes readily. ManNAc is a constituent of numerous glycolipids and glycoproteins, and is the first committed precursor for the biosynthesis of N-acetylneuraminic (Neu5Ac, or sialic acid), which consists of N-acetyl-D-mannosamine in an ether linkage with D-pyruvic acid. ManNAc is formed from UDP-N-acetylglucosamine (UDP-GlcNAc) by the action of UDP-GlcNAc 2-epimerase. ManNAc is then phosphorylated by a specific kinase to ManNAc-6-P (FIG. 1). ManNAc is situated in the sialic acid biosynthesis pathway after the regulated, rate-limiting GNE step (FIG. 1), so its metabolism is not subject to feedback inhibition. Residual ManNAc kinase activity in HIBM patients, or ancillary kinases such as GlcNAc kinase (Hinderlich et al. Eur. J. Biochem. 252: 133-139 (1998)), might convert ManNAc into ManNAc-6-phosphate for subsequent synthesis of sialic acid. In fact, hyposialylated, Gne-deficient mouse embryonic stem cells became resialylated after their growth medium was supplemented with ManNAc (Schwarzkopf et al. Proc. Natl. Acad. Sci. U.S.A. 99: 5267-70 (2002)). Furthermore, incubation of cultured cells with “unnatural” ManNAc derivatives, i.e., N-levulinoylmannosamine (ManLev) or N-azidoacetyl-mannosamine (ManNAz), resulted in incorporation of the downstream sialic acid analogs (SiaLev or SiaNAz) into cell surface glycoconjugates (Charter et al. Glycobiology 10: 1049-56 (2000)).

Studies of an Iranian-Jewish genetic isolate (Argov, et al., Neurology 60, 1519-1523 (2003)) indicate that the autosomal recessive disorder HIBM is mapped to chromosome 9p12-13. The causative gene for HIBM is GNE, coding for the bifunctional enzyme UDP-N-acetylglucosamine-2-epimerase/N-acetylmannosamine kinase. Eisenberg, et al. Nat Genet. 29, 83-87 (2001); Tanner, M. E., Bioorg Chem 33, 216-228 (2005); Stasche, et al. J Biol Chem 272, 24319-24324 (1997); Hinderlich, et al. J Biol Chem 272, 24313-24318 (1997); Jacobs, et al. Biochemistry 40, 12864-12874 (2001). The function and feedback regulation of GNE is depicted in FIG. 1. Distal Myopathy with Rimmed Vacuoles (DMRV, or Nonaka myopathy) is a Japanese variant, allelic to HIBM. Nishino et al. Neurology 59, 1689-1693 (2002); Kayashima et al. J Hum Genet. 47, 77-79 (2002); Hinderlich et al. Neurology 61, 145 (2003). Nearly eighty GNE mutations have been reported in HIBM patients from different ethnic backgrounds, with founder effects among the Iranian Jews and Japanese. Huizing et al, Biochim Biophys Acta 1792, 881-887 (2009); Broccolini et al. Hum Mutat 23, 632 (2004); Eisenberg, et al. Hum Mutat 21, 99 (2003); Tomimitsu, et al. Neurology 59, 451-454 (2002); Darvish, et al. Mol Genet Metab 77, 252-256 (2002). The mutations causing HIBM occur in the regions encoding either the epimerase domain or the kinase domain. Most are missense mutations and result in decreased enzyme GNE activity and underproduction of sialic acid. Sparks et al. Glycobiology 15, 1102-1110 (2005); Penner, et al. Biochemistry 45, 2968-2977 (2006); Huizing and Krasnewich, Biochim Biophys Acta 1792, 881-887 (2009).

Sialic acids are negatively charged terminal sugar moieties added during the post-translational modification on oligosaccharide chains of proteins and lipids to create glycoproteins and glycolipids. Varki, Faseb J 11, 248-255 (1997); Varki et al. Anal Biochem 137, 236-247 (1984). They act as molecular determinants of specific biological processes such as cellular adhesion, cell-cell interactions and signal transduction. Schauer, Glycoconj J 17, 485-499 (2000); Kelm et al. Int Rev Cytol 175, 137-240 (1997).

The pathophysiology of HIBM remains largely unknown, but the dysfunction in GNE suggests that impaired sialylation of glycoproteins is involved. Such a defect could influence cell-cell interactions, intracellular trafficking, organelle biogenesis, apoptosis and secretion. In fact, UDP-GlcNAc 2-epimerase regulates sialylation of cell surface molecules (Keppler et al. Science 284: 1372-76 (1999)), and sialylation appears to be critical for mouse development (Schwarzkopf et al. Proc Natl Acad Sci USA 99, 5267-5270 (2002)).

One hypothesis for the pathophysiology of HIBM involves undersialylation of α-dystroglycan (α-DG), an essential component of the dystrophin-glycoprotein complex. Huizing et al, Mol Genet Metab81, 196-202 (2004); Michele et al. Nature 418, 417-422 (2002); Michele et al. J Biol Chem 278, 15457-15460 (2003). α-DG is heavily glycosylated with O-mannosyl glycans (mannose-N-acetylglucosamine-galactose-sialic acid) linked to a serine or threonine; these glycans are critical for α-DG's interactions with laminin and other extracellular ligands. Aberrant glycosylation of α-DG is the underlying biochemical defect in several congenital muscular dystrophies, generally termed “dystroglycanopathies,” including Fukuyama's congenital muscular dystrophy, Muscle-Eye-Brain disease, Walker-Warburg syndrome and the congenital muscular dystrophies type C1C and C1D. Martin et al., Glycobiology 13, 67R-75R (2003); Martin-Rendon et al. Trends Pharmacol Sci 24, 178-183 (2003). The inventors and others have shown variable hyposialylation of α-DG and other glycoproteins, such as Neural Crest Adhesion Molecule (NCAM), in HIBM. Huizing et al. Mol Genet Metab 81, 196-202 (2004); Savelkoul et al. Mol Genet Metab 88, 389-390 (2006); Sparks et al. BMC Neurol 7, 3 (2007); Broccolini et al. Neuromuscul Disord 15, 177-184 (2005); Ricci et al. Neurology 66, 755-758 (2006); Salama et al. Biochem Biophys Res Commun 328, 221-226 (2005); Tajima et al. Am J Pathol 166, 1121-1130 (2005). A Gne gene-targeted knockin mouse model mimicking the GNE M712T mutation of Iranian-Jewish HIBM patients has been developed as described, for example, in US 2010/0249047, which is incorporated herein by reference in its entirety.

The N-acetylmannosamine (and/or a derivative thereof)-liposome composition, or N-acetylneuraminic acid (and/or a derivative thereof)-liposome composition, disclosed herein may be administered so as to achieve a reduction in at least one symptom associated with an indication or disease. For example, administration of N-acetylmannosamine and/or derivatives thereof can lead to a reduction in proteinuria (e.g., lower amounts of protein in the urine), a reduction in hematuria (e.g., lower amounts of red blood cells in the urine) and improvement of muscle function (e.g., in patients with muscular atrophy).

To achieve the desired effect(s), the N-acetylmannosamine (and/or a derivative thereof)-liposome composition, or N-acetylneuraminic acid (and/or a derivative thereof)-liposome composition, disclosed herein may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 200 to 400 mg/kg or at least about 1 mg/kg to about 25 to 200 mg/kg of body weight, although other dosages may provide beneficial results. The amount administered will vary depending on various factors including, but not limited to the disease, the weight, the physical condition, the health, the age of the mammal, whether prevention or treatment is to be achieved. Such factors can be readily determined by the clinician employing animal models or other test systems that are available in the art.

Administration of the N-acetylmannosamine (and/or a derivative thereof)-liposome composition, or N-acetylneuraminic acid (and/or a derivative thereof)-liposome composition disclosed herein may be in a single dose, in multiple doses, in a continuous or intermittent manner, depending, for example, upon the recipient's physiological condition, whether the purpose of the administration is therapeutic or prophylactic, and other factors known to skilled practitioners. The administration of the N-acetylmannosamine (and/or a derivative thereof)-liposome composition, or N-acetylneuraminic acid (and/or a derivative thereof)-liposome composition, disclosed herein may be essentially continuous over a pre-selected period of time or may be in a series of spaced doses. Both local and systemic administration is contemplated. In certain embodiments, intravenous administration of the liposome compositions once a month may be beneficial.

The N-acetylmannosamine (and/or a derivative thereof)-liposome composition, or N-acetylneuraminic acid (and/or a derivative thereof)-liposome composition, is synthesized or otherwise obtained, and purified as necessary or desired. The absolute weight of N-acetylmannosamine and/or its derivatives, or N-acetylneuraminic acid (and/or a derivative thereof), that is included in a unit dose can vary widely. For example, about 0.01 to about 2 g, or about 0.1 to about 1 g of N-acetyl mannosamine and/or derivatives thereof, or N-acetylneuraminic acid (and/or a derivative thereof), are often used in compositions. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of N-acetylmannosamine and/or derivatives thereof, or N-acetylneuraminic acid (and/or a derivative thereof), can vary as well. Such daily doses can range, for example, from about 0.1 g/day to about 50 g/day, from about 0.2 g/day to about 25 g/day, from about 0.3 g/day to about 12 g/day, from about 0.4 g/day to about 10 g/day, from about 0.5 g/day to about 8 g/day, and from about 0.7 g/day to about 6 g/day.

Thus, one or more suitable unit dosage forms comprising the N-acetylmannosamine (and/or a derivative thereof)-liposome composition, or N-acetylneuraminic acid (and/or a derivative thereof)-liposome composition, can be administered by a variety of routes including oral, parenteral (including subcutaneous, intravenous, intramuscular and intraperitoneal), rectal, dermal, transdermal, intrathoracic, intrapulmonary and intranasal (respiratory) routes.

When the N-acetylmannosamine (and/or a derivative thereof)-liposome composition, or N-acetylneuraminic acid (and/or a derivative thereof)-liposome composition, is prepared for oral administration, it is generally combined with a pharmaceutically acceptable carrier, diluent or excipient to form a pharmaceutical formulation, or unit dosage form. By “pharmaceutically acceptable” it is meant a carrier, diluent, excipient, and/or salt that is compatible with the other ingredients of the formulation, and not deleterious to the recipient thereof.

Pharmaceutical formulations containing the N-acetylmannosamine (and/or a derivative thereof)-liposome composition, or N-acetylneuraminic acid (and/or a derivative thereof)-liposome composition, can be prepared by procedures known in the art using well-known and readily available ingredients. For example, the N-acetylmannosamine (and/or a derivative thereof)-liposome composition, or N-acetylneuraminic acid (and/or a derivative thereof)-liposome composition, can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the N-acetylmannosamine (and/or a derivative thereof)-liposome composition, or the N-acetylneuraminic acid (and/or a derivative thereof)-liposome composition, can include buffering agents such as calcium carbonate, magnesium oxide and magnesium carbonate. Caplets and tablets can also include inactive ingredients such as cellulose, pre-gelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing the N-acetylmannosamine (and/or a derivative thereof)-liposome composition, or the N-acetylneuraminic acid (and/or a derivative thereof)-liposome composition, can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing the N-acetylmannosamine (and/or a derivative thereof)/liposome composition, or the sialic acid/liposome composition, are designed to resist disintegration in the stomach and dissolve in the more neutral to alkaline environment of the duodenum.

The N-acetylmannosamine (and/or a derivative thereof)-liposome composition, or the N-acetylneuraminic acid (and/or a derivative thereof)-liposome composition, can also be formulated as an elixir or solution for convenient oral administration or as a solution appropriate for parenteral administration, for instance by intramuscular, subcutaneous, intraperitoneal or intravenous routes. The pharmaceutical formulations of the N-acetylmannosamine (and/or a derivative thereof)/liposome composition, or the sialic acid/liposome composition, can also take the form of an aqueous or anhydrous solution or dispersion, or alternatively the form of an emulsion or suspension or salve.

Thus, the N-acetylmannosamine (and/or a derivative thereof)-liposome composition, or the N-acetylneuraminic acid (and/or a derivative thereof)-liposome composition, may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The N-acetylmannosamine (and/or a derivative thereof)-liposome composition, or the N-acetylneuraminic acid (and/or a derivative thereof)-liposome composition, and other ingredients may form suspensions, solutions, or emulsions in oily or aqueous vehicles, and may contain formulatory agents such as suspending, stabilizing and/or dispersing agents.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add other ingredients such as antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

For topical administration, the N-acetylmannosamine (and/or a derivative thereof)-liposome composition, or the N-acetylneuraminic acid (and/or a derivative thereof)-liposome composition, may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the N-acetylmannosamine (and/or a derivative thereof)-liposome composition, or the N-acetylneuraminic acid (and/or a derivative thereof)-liposome composition, can be delivered via patches or bandages for dermal administration. Alternatively, the N-acetylmannosamine (and/or a derivative thereof)-liposome composition, or the N-acetylneuraminic acid (and/or a derivative thereof)-liposome composition, can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The N-acetylmannosamine (and/or a derivative thereof)-liposome composition, or the N-acetylneuraminic acid (and/or a derivative thereof)-liposome composition, can also be delivered via iontophoresis, e.g., as disclosed in U.S. Pat. Nos. 4,140,122; 4,383,529; or 4,051,842. The percent by weight of the composition present in a topical formulation will depend on various factors, but generally will be from 0.01% to 95% of the total weight of the formulation, and typically 0.1-85% by weight.

Drops, such as eye drops or nose drops, may be formulated with the N-acetylmannosamine (and/or a derivative thereof)-liposome composition, or the N-acetylneuraminic acid (and/or a derivative thereof)-liposome composition, thereof in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The N-acetylmannosamine (and/or a derivative thereof)-liposome composition, or the N-acetylneuraminic acid (and/or a derivative thereof)-liposome composition, may further be formulated for topical administration in the mouth or throat. For example, the N-acetylmannosamine (and/or a derivative thereof)-liposome composition, or the N-acetylneuraminic acid (and/or a derivative thereof)-liposome composition, may be formulated as a lozenge further comprising a flavored base, usually sucrose and acacia or tragacanth; pastilles comprising the composition in an inert base such as gelatin and glycerin or sucrose and acacia; and mouthwashes comprising the presently disclosed compositions in a suitable liquid carrier.

The pharmaceutical formulations may include, as optional ingredients, pharmaceutically acceptable carriers, diluents, solubilizing or emulsifying agents, and salts of the type that are available in the art.

Furthermore, the N-acetylmannosamine (and/or a derivative thereof)-liposome composition, or the N-acetylneuraminic acid (and/or a derivative thereof)-liposome composition, may also be used in combination with other therapeutic agents, for example, pain relievers, anti-inflammatory agents, and the like, whether for the conditions described or some other condition.

Also disclosed herein is a packaged pharmaceutical composition such as a kit or other container for increasing production of sialic acid in a mammal. The kit or container holds a therapeutically effective amount of a pharmaceutical composition for increasing intracellular production of sialic acid and instructions for using the pharmaceutical composition for increasing production of sialic acid in the mammal. The pharmaceutical composition includes the N-acetylmannosamine (and/or a derivative thereof)-liposome composition, or the N-acetylneuraminic acid (and/or a derivative thereof)-liposome composition, in a therapeutically effective amount such that sialic acid production is increased.

Several embodiments are described in the following numbered paragraphs:

1. A method for treating a disorder characterized by hyposialylation in a subject comprising administering to the subject an effective amount of (i) a composition comprising N-acetylmannosamine, or a derivative thereof, encapsulated within a liposome, or (ii) a composition comprising N-acetylneuraminic acid, or a derivative thereof, encapsulated within a liposome, wherein the liposome comprises 1,2-dioleoyl-3-trimethylammonium-propane and cholesterol.

2. A method for increasing sialic acid in a subject in need thereof comprising administering to the subject an effective amount of (i) a composition comprising N-acetylmannosamine, or a derivative thereof, encapsulated within a liposome, or (ii) a composition comprising N-acetylneuraminic acid, or a derivative thereof, encapsulated within a liposome, wherein the liposome comprises 1,2-dioleoyl-3-trimethylammonium-propane and cholesterol, to thereby increase sialic acid levels in the subject.

3. A method for treating a disorder characterized by hyposialylation in a subject comprising:

identifying a subject in need of treatment for hyposialylation; and

administering to the subject an effective amount of (i) a composition comprising N-acetylmannosamine, or a derivative thereof, encapsulated within a liposome, or (ii) a composition comprising N-acetylneuraminic acid, or a derivative thereof, encapsulated within a liposome, wherein the liposome comprises 1,2-dioleoyl-3-trimethylammonium-propane and cholesterol.

4. The method of any one of paragraphs 1, 2 or 3, wherein the composition (i) or the composition (ii) is administered to the subject intravenously.

5. The method of any one of paragraphs 1, 2 or 3, wherein composition (i) is administered to the subject.

6. The method of any one of paragraphs 1, 2 or 3, wherein composition (ii) is administered to the subject.

7. The method of any one of paragraphs 1, 3, 4, 5 or 6, wherein the disorder is myopathy, a kidney condition, a hyposialylated cancer, or a neurodegenerative disease.

8. The method of paragraph 7 wherein the myopathy is hereditary inclusion body myopathy.

9. The method of paragraph 7 wherein the myopathy is distal myopathy with rimmed vacuoles.

10. The method of any one of paragraphs 1, 2, 3, 4, 5, 7, 8 or 9, wherein composition (i) comprises N-acetylmannosamine.

11. The method of any one of paragraphs 1, 2, 3, 4, 6 7, 8 or 9, wherein composition (ii) comprises N-acetylneuraminic acid.

### EXAMPLES

In the experiments described below, the monosaccharides N-acetyl-D-mannosamine (ManNAc) and N-acetylneuraminic acid (Neu5Ac, NANA) were encapsulated in a dioleoyl trimethylammonium propane (DOTAP): cholesterol liposome. Injection of these liposome formulations into mice showed efficient delivery to affected tissues.

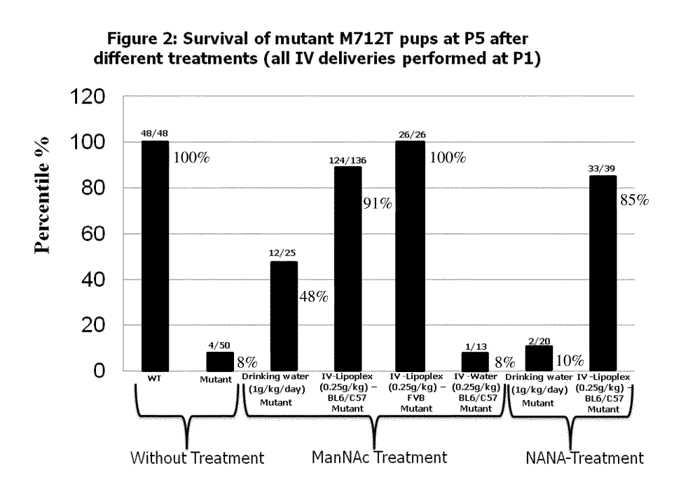

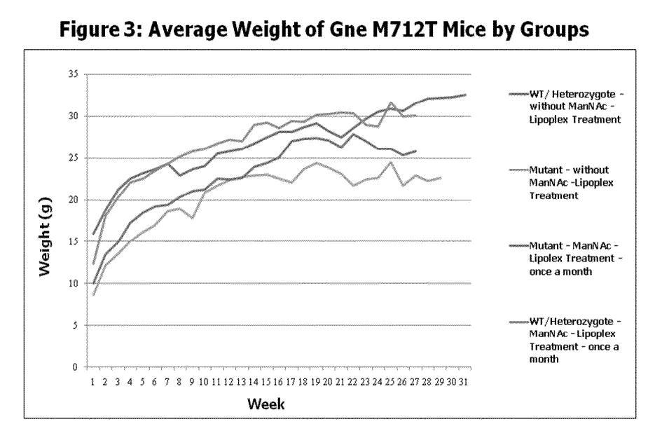

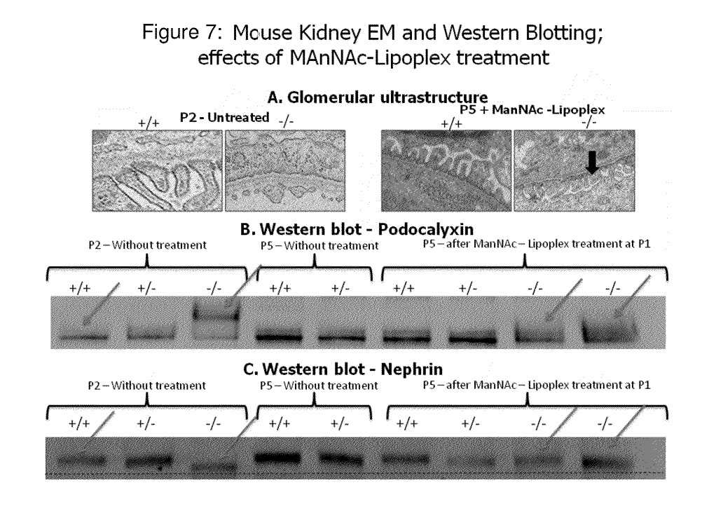

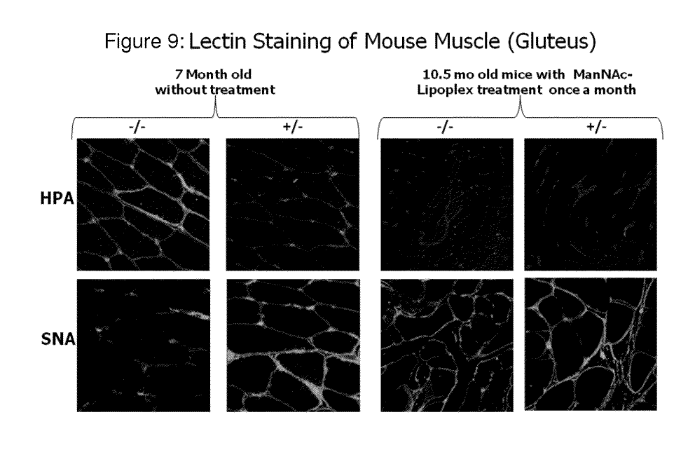

In particular, ManNAc or NANA formulated inside a liposome (ManNAc-Lipoplex, NANA-Lipoplex) can be used to treat symptoms of hyposialylation in a mouse model of HIBM (mice with Gne M712T knock-in mutation). This model mimics the human M712T mutation, common in the Persian-Jewish population, in the key enzyme of sialic acid synthesis: UDP-N-acetylglucosamine-2-epimerase/ManNAc kinase (GNE). Without treatment, more than 90% of mutant M712T mice (−/−) die before postnatal day 3 (P3) due to severe glomerulopathy (kidney failure) caused by hyposialylation. In our studies, intravenous ManNAc-Lipoplex and NANA-Lipoplex injections at P1 yielded survival beyond P3 in over 90% of the −/− pups (FIG. 2), compared to much lower survival rates when orally delivered in drinking water (48% for ManNAc, 10% for NANA). In addition, the weights (and thus muscle mass) of the −/− survivors continuously treated with ManNAc-Lipoplex until adulthood were very similar to those of untreated heterozygous and wild type animals, indicating rescue of muscle degeneration and/or rescued kidney function with treatment (FIG. 3). Finally, lectin staining and western blotting of sialylated proteins showed that ManNAc-Lipoplex and NANA-Lipoplex treatment resulted in rescue of hyposialylation in the kidneys (FIGS. 5, 6, 7 and 8) and muscles (FIG. 9) of −/− pups. In these experiments, ManNAc-Lipoplex and NANA-Lipoplex was delivered intravenously, and we believe that it was able to cross the blood-brain barrier more efficiently than oral or intravenous delivery of ManNAc (or NANA) alone (without microencapsulation with liposomes).

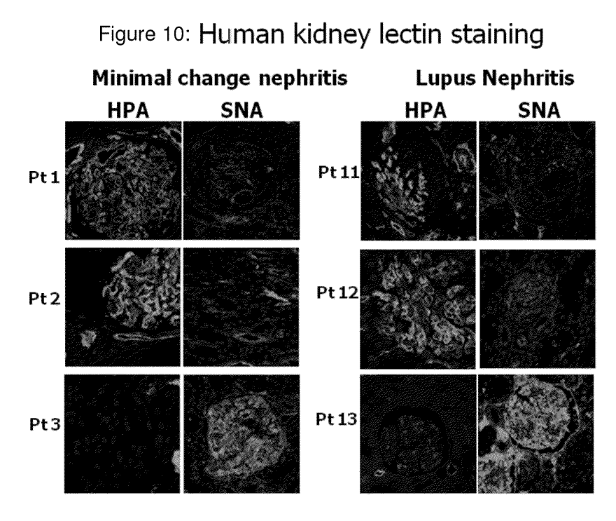

Experiments on human samples were also performed to detect hyposialylation. Forty human kidney biopsy and/or autopsy slides of patients with a variety of renal disorders mimicking the Gne M712T mouse model (hematuria and/or proteinuria, glomerular disease) were tested with a lectin staining panel. Eight of these kidneys were found to have severe glomerular hyposialylation similar to the Gne M712T mouse model (summarized in FIG. 10). This indicates that these kidney disorders may benefit from ManNAc-Lipoplex or NANA-Lipoplex therapy. The tested human renal disorders included minimal change nephrosis, lupus nephritis, IgA nephropathy, focal segmental glomerulosclerosis and glomerulonephritis.

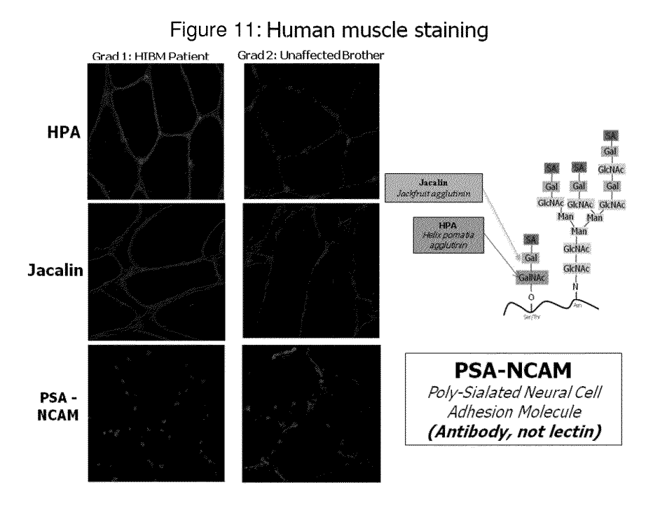

In addition, hyposialylation of muscle biopsies from HIBM patients was shown by lectin staining and staining for the sialylated glycan polysialic acid on neural crest adhesion molecule (PSA-NCAM) (FIG. 11). This is a further indication that HIBM patients may benefit from ManNAc-lipoplex or NANA-lipoplex therapy.

### ManNAc-Lipoplex and NANA-Lipoplex

Sterile pharmaceutical (cGMP) grade liposomes comprising ManNAc or Sialic acid (Neu5Ac) in DOTAP:Cholesterol liposomes (ManNAc-Lipoplex or NANA-Lipoplex) for systemic delivery were prepared as described below.

Reagents and Chemicals: ManNAc and NANA were obtained from New Zealand Pharmaceuticals, DOTAP and Cholesterol were obtained from Avanti, HPLC grade Chloroform and USP grade water were also used. Briefly, 280±2% mg DOTAP and 138.6±2% mg Cholesterol powder was weighed out, transferred to a glass vessel, and dissolved in 50 ml chloroform and dried. 10 ml ManNAc or NANA containing water was used to re-hydrate the lipids (222.26±2% mg sugar/ml 5× Liposome). The liposomes were sonicated, then sterile-filtered through successively smaller syringe filters, and sized. 1 ml final product was aliquotted per vial into 1.2 ml cryovials.

TABLE 1

| | ManNAc - | ManNAc - | NANA - | NANA - |
|---|---|---|---|---|
| | Liposomes | water | Liposomes | water |
| DOTAP | 140.2 mg | n/a | 140.2 mg | n/a |
| mass | | | | |
| Chol mass | 69.4 mg | n/a | 69.4 mg | n/a |
| ManNAc | 2223.2 mg | 2223.2 mg | 2222.9 mg | 2222.9 mg |
| mass | | | | |
| Dose | 222.32 mg/ml | 222.32 mg/ml | 222.29 mg/ml | 222.29 |
| | ManNAc in 5x | ManNAc | NANA in 5x | mg/ml |
| | Liposomes | in water | Liposomes | NANA |
| | in water | | in water | in water |

The stock concentration of the 5×ManNAc-Lipoplex was 333 mg/ml in USP grade D5W water. The stock concentration of the 5×NANA-Lipoplex was 222 mg/ml in USP grade water. All injections for P1 HIBM Gne M712T pups were performed with 1.6 μl (0.5 mg) of ManNAc-Lipoplex dissolved in 6.4 μl USP grade water or 4.8 μl (0.5 mg) of NANA-Lipoplex dissolved in 3.2 μl USP grade water (injection method for P1 pup described in Yardeni et al, Lab Animals 40, 155-160 (2011)). For adult mice all injections were made in 6 μl (1 mg) of ManNAc-Lipoplex dissolved in 24 μl USP grade water.

### HIBM Mouse Model

Gne^{M712T/M712T} knockin mice were generated by targeting the p. M712T (ATG to ACG) mutation of exon 12 of the murine Gne gene (Gne, Uael, GenBank NM_{—}015828) as previously described (Galeano et al., J. Clin. Invest. 117, 1585:1594 (2007)). The mutant mice were maintained in the C57BL/6J and FVB backgrounds. Animals were housed in an Association for Assessment and Accreditation of Laboratory Animal Care International-accredited specific pathogen-free facility in accordance with the Guide for the care and use of laboratory animals (NIH publication no. 85-23). Cages were ventilated in a temperature- and light-controlled environment (22° C., 30%-70% humidity, 12-hour light/12-hour dark cycle). The mice were fed irradiated chow (Prolab 5P75 Isopro 3000; PMI Nutrition International) and sterile water and libitum. All mouse procedures were performed in accordance with NIH Animal Protocol #G-04-03 “Development of Mouse Models for Hereditary Inclusion Body Myopathy (HIBM)” approved by the Institutional Animal Care and Use Committee of the National Human Genome Research Institute.

All euthanasia was performed by CO₂ inhalation followed by cervical dislocation. Untreated pups were euthanized at postnatal day 1 (P1), P2 (since mutant pups died before P3) and P5 (for wild type and heterozygote pups). Treated pups were euthanized at P5 or grown into adulthood. Tail snips were collected from all mice for genotyping, tissues were collected for fixation or storage in −80° c. for further study and urine were collected for proteinuria test. For embryonic tissue collection, pregnant mice at E17 were euthanized, and embryos were retrieved by cesarean section and euthanized by decapitation. For higher percentages of mutants per experiment, we mated heterozygous female mice with mutant male mice; these mating did not yield any wild type pups. We studied heterozygous rather than wild type pups for some (treatment) experiment.

### ManNAc-lipolex and NANA-Lipoplex Treatments

ManNAc was received from New Zealand Pharmaceuticals, (Palmerston North, New Zealand). ManNAc treatment was performed by retro-orbital injection of ManNAc encapsulated in liposomes (ManNAc-Lipoplex) to whole litters at P1, part of the treated mice received additional ManNAc-Lipoplex treatment at older age. Sialic acid (Neu5Ac) was received from New Zealand Pharmaceuticals (Palmerston North, New Zealand). Sialic acid treatment was performed by retro-orbital injection of NANA-Lipoplex encapsulated in liposomes (NANA-Lipoplex) to whole litters at P1.

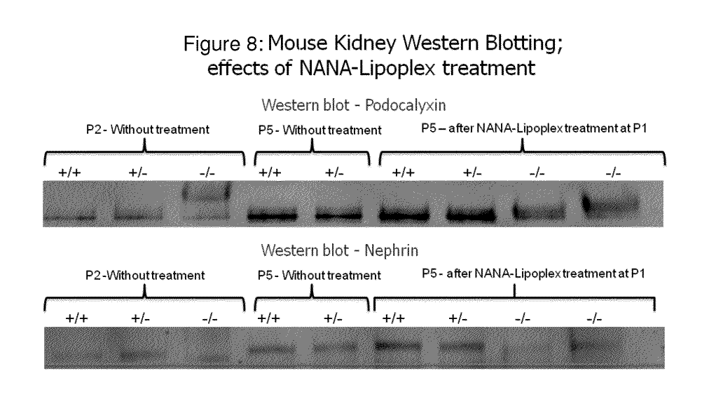

Retro-Orbital (IV) Injection at Postnatal Day 1(P1) or for Adult Mice Intravenous injections of neonatal mice are very challenging, since the tail vein at P1 cannot be injected. In these examples, we developed a novel technique for intra-venous (IV) delivery by retro—orbital injection of newborn pups. For these retro-orbital injections, a 31 gauge, 5/16 inch needle attached to a 0.3 ml insulin syringe was used (BD Ultra-Fine II™, Becton, Dickinson and Co., Franklin Lakes, N.J.). All injections at Plwere done in a total volume of 8 μl. The pups were not anesthetized for this procedure. The injections were performed with the aid of a dissecting microscope (8 to 10× magnification is usually sufficient). Before injection the pups were placed in a small container that included a protected warming device and soft gauze (Nu Gauze®, Johnson & Johnson, New Brunswick, N.J.). Once the pups were placed in the container, an additional gauze pad covered them to provide added warmth. The pup was placed in right lateral recumbency with the pup's head facing to the right so that the right retro-orbital sinus could be injected. The pup's head was gently restrained with the tip of the thumb and forefinger. Caution was used with the restraint the trachea or impede venous flow was not collapsed. The rest of the pup's body was nestled between thumb and forefinger. The area over the eye was gently cleaned with sterile saline and a cotton-tipped applicator. This helped to remove any skin flakes that could get in the way of the injection and helps to make the skin slightly more transparent. The needle was then inserted, bevel down, at the 3 o'clock position into the eye socket (i.e., the area that would become the medial canthus) at approximately a 30-degree angle (FIGS. 8B&8D). The back of the socket was mentally visualized and the needle was advanced to the area of the retro-orbital sinus. The injection was made in a gentle, smooth, fluid motion. Once the injection was complete, the needle was slowly and smoothly withdrawn. After which, the pups were returned to their home cage with their mother.

For retro-orbital injections in adult mice, a 27.5 gauge or smaller, 0.5 inch insulin needle and syringe (Terumo® U-100, Terumo Medical Corp., Elkton, Md.) was used. The injectate volumes were 30-80 μl. The mice were anesthetized for this procedure with an inhalant anesthetic because of its rapid induction and recovery. Using isoflurane, we induce our mice in a plexiglass chamber and then placed them on a funnel-shaped nose cone connected to a non-rebreathing apparatus for the injection. Waste anesthetic gases were managed with a down draft table and/or a charcoal scavenging device. The mice were injected to the right retro-orbital sinus. The mouse was placed in right lateral recumbence so that its head faced to the right. The eyeball was then partially protruded from the eye socket by applying gentle pressure to the skin dorsal and ventral to the eye. In addition to the inhalant anesthetic, a drop of ophthalmic anesthetic (0.5% proparacaine hydrochloride ophthalmic solution, Alcon Laboratories, Fort Worth, Tex.) was placed on the eye to be injected. The needle was carefully introduced, bevel down (to decreased the likelihood of damaging the eyeball), at approximately a 30-degree angle into the medial canthus. Once the injection was complete, the needle was slowly and smoothly withdrawn. The animal was then placed back into its cage for recovery.

### Mouse Kidney and Muscle Histology

Mouse kidneys or muscles were fixed in 4% paraformaldehyde for 48 hours, dehydrated in 70% ethanol, and paraffin embedded for sectioning. Tissue sections were subjected to (immuno-) histochemistry with a variety of antibodies and lectins.

### Electron Microscopy

For transmission EM, mouse kidney samples were fixed overnight at 4° C. in 2% glutaraldehyde in 0.1M cacodylate buffer (pH 7.4) and washed with cacodylate buffer. The tissues were then fixed with 2% OsO₄ for two hours, washed again with 0.1 M cacodylate buffer, washed with water and placed in 1% uranyl acetate for one hour. The tissues were then serially dehydrated in ethanol and propylene oxide and embedded in EMBed 812 resin (Electron Microscopy Sciences, Hatfield, Pa., USA). Thin sections (˜80 nm) were obtained using a Leica Ultracut-UCT Ultramicrotome (Leica Microsystems, Deerfield, Ill.), placed onto 300 mesh copper grids and stained with saturated uranyl acetate in 50% methanol followed by lead citrate. The grids were viewed in a JEM-1200EXII electron microscope (JEOL Ltd, Tokyo, Japan) at 80 kV, and images were recorded on a XR611M, mid mounted, 10.5 Mpixel, CCD camera (Advanced Microscopy Techniques Corp, Danvers, Mass.).

### Lectin and Antibodies Histochemistry

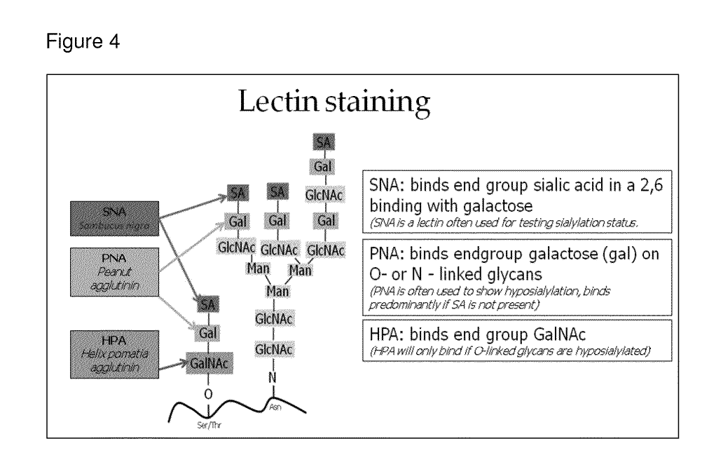

For lectin histochemistry, the FITC labeled lectins PNA (peanut agglutinin from Arachis hypogaeae), Jacalin (jackfruit agglutinin from Artocarpus integrifolia), HPA (edible snail agglutinin from Helix pomatia), and SNA (elderberry bark from Sambucus nigra) were obtained from EY Laboratories (San Mateo, Calif.). Their glycan specific is indicated in FIG. 4. In general, SNA predominantly bind sialic acid residues on glycans, while PNA and Jacalin predominantly bind end group galactose residues (when sialic acid is absent), and predominantly binds O-linked GalNAc (without other residues a trained). Paraffin embedded kidney or muscle sections (human and mouse) were deparaffinized and rehydrated, followed by antigen retrieval in 0.01M sodium citrate (pH 6.4) and blocking in carbohydrate free blocking solution (Vector laboratories, Burlingame, Calif.) for 1 hour at room temperature (RT). The slides were then incubated at 4° C. overnight with each lectin aliquoted in carbohydrate free blocking solution (FITC-HPA 5 μg/ml, FITC-WGA 15 μg/ml, FITC-PNA 30 μg/ml, Jacalin 50 μg/ml, FITC-SNA 5 μg/ml and FITC-VVA 5 μg/ml). Washes were performed with 0.1% Triton-X-100 in 1×Tris-buffered saline (TBS). The PNA, Jacalin and HPA stained slides were incubated in 0.3% sudan black in 70% ethanol solution for 10 minutes to reduce autofluorescence. In addition, to verify lectin specificity, each lectin was incubated with its specific inhibitory carbohydrate for 1 hour prior to overnight incubation on a slide. The used inhibitory carbohydrates were Neu5Ac (Toronto Research Chemicals, North York, Ontario, Canada) for SNA, lactose (C-6010-10, EY Laboratories) for PNA, galactose (C-6003-10, EY Laboratories) for Jacalin, and GalNAc (C-6000-10, EY Laboratories) for HPA.

For antibodies (podocalyxin, nephrin) immunohistochemistry, paraffin embedded sections were deparaffanized and rehydrated, followed by antigen retrieval in 0.01M sodium citrate (pH 6.4) and blocking in 1% BSA/2% Triton-X solution for 4 hours at RT. Goat-anti-mouse podocalyxin antibodies (1:200; AF1556, R&D Systems, Minneapolis, Minn.) or Guinea-pig-anti-mouse Nephrin (1:100; GP-N2, PROGENE Biotechnik, Heidelberg, Germany) were incubated at 4° C. overnight, followed by incubation with chicken-anti-goat Alexa Fluor 488 for podocalyxin and gout-anti-Guinea-pig Alexa Fluor 488 for Nephrin, for 1 hour RT (Molecular Probes, Carlsbad, Calif.). All slides were subsequently rinsed and mounted in Vectashield including the nuclear stain DAPI (H-1200, Vector Laboratories, Burlingame, Calif., USA). All fluorescence imaging was performed on a Zeiss 510 META confocal laser-scanning microscope (Zeiss, Thornwood, N.Y.). All images represent collapsed stacks of confocal Z-sections, imaged with a 63× objective for mouse kidney, or 40× for human kidney or mouse muscle.

### Western Blot Analysis

Frozen kidney specimens were homogenized in CelLytic buffer consisting of a mild detergent, bicine buffer, and 150 mM NaCl (Sigma-Aldrich, St Louis, Mo.) supplemented with protease inhibitors (Complete Mini; Roche Applied Science, Indianapolis, Ind.). The lysates were sonicated and cleared by centrifugation (1,000 g for 10 minutes), and the resulting supernatants were assayed for protein (BCA Protein Assay; Pierce Biotechnology, Rockford, Ill.). Equal amounts of protein (10-30 μg) were electrophoresed on 4%-20% Tris-Glycine gels (Novex; Invitrogen, Carlsbad, Calif.) and electroblotted onto 0.45 μm Hybond ECL nitrocellulose membranes (GE Health Care, Piscataway, N.J.). For podocalyxin and Nephrin immunoblotting, membranes were blocked (10% fat-free milk) and incubated with primary anti-podocalyxin or anti-Nephrin antibodies (podocalyxin; AF1556, R&D Systems, Minneapolis, Minn. Nephrin; GP-N2, PROGENE Biotechnik, Heidelberg, Germany) or mouse monoclonal α-tubulin antibodies (loading control, Sigma-Aldrich, St. Louis, Mo.), followed by HRP-conjugated secondary antibodies (GE HealthCare, Piscataway, N.J. and Santa Cruz Biotechnology, Santa Cruz, Calif.). Results were visualized with enhanced chemiluminescence (ECL Western Blotting Detection Reagents; GE HealthCare, Piscataway, N.J.) and exposure to CL-XPosure Film (Pierce Biotechnology, Rockford, Ill.).

### Adult Mouse Muscle Phenotype Characterization

HIBM is adult onset, therefore mutant pups who received ManNAc-Lipoplex at P1, were grown (without further treatment) into adulthood. The mice were divided into groups (see Table 2 below), untreated or treated with ManNAc-Lipoplex), 3 different injections time points, for both mutant and wild type/heterozygote groups. The mice were weighed once a week. Mice were euthanized at different ages to biochemically check the progression and the symptoms of disease onset. Tissues were collected for further characterization by histology and biochemical analysis.

TABLE 2

| | | ManNAc-Lipoplex |
|---|---|---|
| Mouse Genotype | Group | Treatment |
| Wild Type/Heterozygote | Ctrl | None |
| M712T mut | Ctrl | None |
| M712T mut | Exp | IV every month |
| M712T mut | Exp | 1 IV |
| M712T mut | Exp | 3 IV |
| Wild Type/Hetrozygote | Ctrl | 3 IV |
| Wild Type/Hetrozygote | Ctrl | IV every month |

In view of the many possible embodiments to which the principles of the disclosed compositions and methods may be applied, it should be recognized that the illustrated embodiments are only preferred examples and should not be taken as limiting the scope of the invention.

What is claimed is:

1. A method for treating a disorder characterized by hyposialylation in a subject comprising administering to the subject an effective amount of (i) a composition comprising N-acetylmannosamine, or a derivative thereof, encapsulated within a liposome, or (ii) a composition comprising N-acetylneuraminic acid, or a derivative thereof, encapsulated within a liposome, wherein the liposome comprises 1,2-dioleoyl-3-trimethylammonium-propane and cholesterol.

2. A method for increasing sialic acid in a subject in need thereof comprising administering to the subject an effective amount of (i) a composition comprising N-acetylmannosamine, or a derivative thereof, encapsulated within a liposome, or (ii) a composition comprising N-acetylneuraminic acid, or a derivative thereof, encapsulated within a liposome, wherein the liposome comprises 1,2-dioleoyl-3-trimethylammonium-propane and cholesterol, to thereby increase sialic acid levels in the subject.

3. A method for treating a disorder characterized by hyposialylation in a subject comprising:
identifying a subject in need of treatment for hyposialylation; and
administering to the subject an effective amount of (i) a composition comprising N-acetylmannosamine, or a derivative thereof, encapsulated within a liposome, or (ii) a composition comprising N-acetylneuraminic acid, or a derivative thereof, encapsulated within a liposome, wherein the liposome comprises 1,2-dioleoyl-3-trimethylammonium-propane and cholesterol.

4. The method of claim 1, wherein the composition (i) or the composition (ii) is administered to the subject intravenously.

5. The method of claim 1, wherein composition (i) is administered to the subject.

6. The method of claim 1, wherein composition (ii) is administered to the subject.

7. The method of claim 1, wherein the disorder is myopathy, a kidney condition, a hyposialylated cancer, or a neurodegenerative disease.

8. The method of claim 7 wherein the myopathy is hereditary inclusion body myopathy.

9. The method of claim 7 wherein the myopathy is distal myopathy with rimmed vacuoles.

10. The method of claim 1, wherein composition (i) comprises N-acetylmannosamine.

11. The method of claim 1, wherein composition (ii) comprises N-acetylneuraminic acid.

12. The method of claim 2, wherein the composition (i) or the composition (ii) is administered to the subject intravenously.

13. The method of claim 2, wherein composition (i) is administered to the subject.

14. The method of claim 2, wherein composition (ii) is administered to the subject.

15. The method of claim 2, wherein composition (i) comprises N-acetylmannosamine.

16. The method of claim 2, wherein composition (ii) comprises N-acetylneuraminic acid.
